(19) United States
(12) Patent Application Publication
(10) Pub. No.: US 2022/0274902 A1
(43) Pub. Date: Sep. 1, 2022

(54) SELECTIVE PRODUCTION OF PROPYLENE AND BUTYLENE FROM METHANE

(71) Applicant: SABIC Global Technologies B.V., Bergen op Zoom (NL)

(72) Inventor: Muhammad H. HAIDER, Riyadh (SA)

(21) Appl. No.: 17/627,037

(22) PCT Filed: Jul. 9, 2020
(86) PCT No.: PCT/IB2020/056466

§ 371 Date: Jan. 13, 2022

Related U.S. Application Data
(60) Provisional application No. 62/875,473, filed on Jul. 17, 2019.

(51) Int. Cl. C07C 29/156; B01J 6/00; B01J 23/882; B01J 23/30; B01J 21/04

(52) U.S. Cl. CPC C07C 29/156; B01J 6/001; B01J 23/882; B01J 23/30; B01J 21/04; C07C 2523/882; C07C 2523/30

(57) ABSTRACT

Disclosed are processes for producing propylene and butylene. The processes can include contacting a first stream containing methane with an oxidant and oxidizing at least a portion of the methane under conditions suitable to produce a second stream containing carbon monoxide (CO) and hydrogen (H₂), contacting the second stream with a CO hydrogenation catalyst under conditions suitable to produce a third stream containing propanol and butanol, and contacting the third stream with an dehydration catalyst under conditions suitable to dehydrate at least a portion of the propanol and butanol and produce a products stream containing propylene and butylene.

### CROSS REFERENCE TO RELATED APPLICATIONS

This application claims the benefit of priority of U.S. Provisional Patent Application No. 62/875,473, filed Jul. 17, 2019, which is hereby incorporated by reference in its entirety.

### FIELD OF INVENTION

The invention generally concerns compositions, processes, and systems for production of propylene and butylene. In particular, the invention concerns compositions, processes, and systems for the selective production of propylene and butylene from methane via converting methane into syngas, producing propanol and butanol from the syngas via carbon monoxide (CO) hydrogenation, and converting the propanol and butanol into propylene and butylene respectively via alcohol dehydration.

### BACKGROUND OF THE INVENTION

Short chain olefins, such as propylene and butylene, production is a global trend in petrochemical industries. This trend has been forecasted to stay for longer periods since industrial processes have started integrating the upstream with downstream processes. The production market of propylene and butylene will increase so as the demand. Methane is a major hydrocarbon obtained from refinery, petrochemical, and renewable substrates. It is largely consumed as burning fuel. However, producing propylene and butylene from methane can be beneficial for petrochemical industries.

There are many types of processes known to produce propylene and butylene. By way of example, dehydrogenation of propane and butane can be used to produce such olefins. Fluid catalytic cracking (FCC) processes can also be used to produce such olefins. Some of the problems associated with these processes is the use of feed stocks that can increase the costs and inefficiencies of producing propylene and butylene.

While other processes exist for producing liquid hydrocarbons (e.g., alkanes) from syngas, these processes do not lead to propylene or butylene production. By way of example, EP2944606A1 discloses forming liquid hydrocarbons and generating H₂ and CO₂ from Fischer-Tropsch off-gas produced in said Fischer-Tropsch process. EP2944606A1, however, fails to disclose selectively producing propanol and butanol from syngas during the Fischer-Tropsch process, much less producing propylene and butylene.

### SUMMARY OF THE INVENTION

A discovery has been made that provides a solution to at least some of the aforementioned problems associated with the production of propylene and butylene. A solution of the present invention can include the conversion of methane to propylene and butylene through a sequence of steps that can result in high selectivity towards propylene and butylene production in a reasonably cost-efficient manner. The sequence of steps can include (a) conversion of methane into carbon monoxide (CO) and hydrogen (H₂) (e.g., syngas), (b) conversion of CO into propanol and butanol via CO hydrogenation, and (c) conversion of propanol and butanol into propylene and butylene respectively via alcohol dehydration. In one particular instance, it was discovered that a cobalt/molybdenum catalyst having a β-phase crystal structure can be used as a CO hydrogenation catalyst, which can produce propanol and butanol with a high selectivity, which can further enhance the efficiency of the overall methane to propylene and butylene conversion process.

In one aspect of the present invention, a process for producing propylene and butylene is described. The process can include steps (a)-(c). In step (a) a first stream containing methane can be contacted with an oxidant and at least a portion of the methane can be oxidized under conditions suitable to produce a second stream containing carbon monoxide (CO) and hydrogen (H₂). In step (b) the second stream can be contacted with a CO hydrogenation catalyst under conditions suitable to produce a third stream comprising propanol and butanol, by hydrogenating the CO with the Hz. In step (c) the third stream can be contacted with a dehydration catalyst under conditions suitable to dehydrate at least a portion of the propanol and butanol and produce a products stream containing propylene and butylene. In some aspects, the third stream can further contain C2-C7 paraffins, methane, and carbon dioxide (CO₂). In some aspects, at least a portion of the C2-C7 paraffins, methane, and carbon dioxide (CO₂) can be separated from the third stream before contacting the third stream with the dehydration catalyst. In some aspects, the CO hydrogenation catalyst can include a cobalt molybdenum containing catalyst having a β-phase crystal structure. In some aspects, the cobalt molybdenum containing catalyst can include a cobalt molybdenum oxide having a β-phase crystal structure. In some aspects, the CO hydrogenation catalyst can comprise a calcined composition comprising: β-CoₓMo_{y}O_{z}, with x ranging from 0.5 to 1.5, preferably 0.9 to 1.1, y ranging from 0.5 to 1.5, preferably 0.9 to 1.1, and z can be a value that balances the valencies of Co and Mo. In certain aspects z can be 3.5 to 4.5, preferably 3.9 to 4.1. In some aspects, the calcined composition can be essentially free of or contain no beta-molybdenum carbide (β-Mo₂C), an alkaline metal promoter, and an alkaline earth metal promoter. In some particular aspects, the calcined composition can comprise β-CoMoO₄. In some aspects, the CO hydrogenation catalyst can be activated, prior to contacting the catalyst with the second stream. The catalyst can be activated by reduction with hydrogen (H₂). In certain instances, the activation process can include reducing the catalyst with a stream containing hydrogen (H₂) at a temperature 200° C. to 500° C., at a GHSV of 1000 to 3000 and/or at pressure 25 bar to 90 bar for 8 h to 20 h. The oxidant in step (a) can be steam, oxygen (O₂), CO₂, or a combination thereof. The oxidation of the methane in step (a) can be catalyzed using a methane oxidation catalyst. In some aspects, the methane oxidation catalyst can include one or more metals on a support. The one or more metals can be one or more of La, Ni, Ru, Rh, Pd, Ir or Pt or any combination or alloy or oxide thereof. The support can be alumina, silica, zirconia, ceria, titania, magnesium oxide, magnesium aluminate, or any combination thereof. In some aspects, the methane oxidation catalyst can contain a promoter. In some aspects the promoter can be an alkali metal and/or an alkaline earth metal. In some aspects the promoter can include Li, Na, K, or any combination or alloy or oxide thereof. The methane oxidation conditions in step (a) can include a pressure of 0 to 180 bar, GHSV of 5000 to 15000 and/or a temperature of 500 to 1600° C. In some aspects, the methane in the first stream can be obtained from a refinery, petroleum by product, or renewable feedstock or combinations thereof. The molar ratio of the H₂ and CO in the second stream can be 0.5:1 to 3:1, preferably 0.8:1 to 1.2:1. The contacting conditions in step (b) can include a pressure of 25 to 90 bar, GHSV of 1000 to 3000 and/or a temperature of 150 to 450° C. In some aspects, in step (b) the CO conversion can be 25% to 35%, propanol selectivity can be 12% to 25% and butanol selectivity can be 20% to 45%. In some aspects, in step (b) propanol selectivity can be 12% to 30%.

The combined mol. % of the propanol and butanol in the third stream can be at least 50 mol. %. In some aspects, the combined mol. % of propanol and butanol in the third stream can be 50 mol. % to 70 mol. %. In some aspects third stream can contain 12 mol. % to 25 mol. %, preferably 15 mol. % to 22 mol. %, propanol; 20 mol. % to 45 mol. %, preferably 30 mol. % to 42 mol. wt. %, butanol; 30 mol. % to 45 mol. %, C2 to C7 paraffins; 3 mol. % to 10 mol. %, methane and 0 to 5 mol. % CO₂. In some aspects, the third stream can be essentially free of or contain no methanol. In some aspects, the third stream can be essentially free of or contain no methanol. Combined selectivity of methanol and ethanol obtained from CO hydrogenation in step (b) can be less than 10%, preferably less than 8%, more preferably less than 5%. In some aspects, selectivity of methanol obtained in step (b) can be less than 5%, preferably less than 3%, more preferably less than 2%. In some aspects, selectivity of ethanol obtained in step (b) can be less than 5%, preferably less than 3%, more preferably less than 2%. In some aspects, C2 to C7 paraffins, methane and CO₂ can be separated from the third stream by traditional gas liquid separation. In some aspects, C2 to C7 paraffins, methane and CO₂ can be separated from the third stream by distillation using a distillation column. The propanol and butanol dehydration conditions in step (c) can include a pressure of 0 to 90 bar, GHSV of 1000 to 3000 and/or a temperature of 105 to 450° C. The dehydration catalyst in step (c) can be an acid type catalyst. In some aspects the acid type catalyst can be cesium doped silicotungstic acid supported on alumina.

The combined wt. % of propylene and butylene in the products stream obtained in step (c) can be 90 wt. % to 100 wt. %, preferably 95 wt. % to 100 wt. %, more preferably 98 wt. % to 100 wt. %. The wt. % of propylene in the products stream obtained in step (c) can be 30 wt. % to 40 wt. %. The wt. % of butylene in the products stream obtained in step (c) can be 60 wt. % to 70 wt. %.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to other aspects of the invention. It is contemplated that any embodiment discussed herein can be implemented with respect to any method or composition of the invention, and vice versa. Furthermore, compositions and systems of the invention can be used to achieve methods of the invention.

The following includes definitions of various terms and phrases used throughout this specification.

C2-C7 paraffins refers to paraffin hydrocarbons having a carbon number 2 to 7 (e.g. ethane, propane, butane, pentane, hexane, heptane etc.).

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %,” “vol. %,” or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%. “Essentially free” is defined as having no more than about 0.1% of a component. % can be relative to wt. %, vol. %, or mol. %.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Selectivity of a compound in a reaction is defined as: Selectivity of a compound one=(moles of compound one produced/total moles produced)*100.

The process and systems of the present invention can “comprise,” “consist essentially of,” or “consist of” particular ingredients, components, compositions, steps, etc. disclosed throughout the specification. With respect to the transitional phrase “consisting essentially of,” in one non-limiting aspect, a basic and novel characteristic of the processes and the systems of the present invention are their abilities to produce propylene and butylene from methane using intermediate steps CO and H₂ formation from methane, propanol and butanol production from CO hydrogenation, and propanol and butanol dehydration to produce propylene and butylene. The CO hydrogenation step can have a relatively high selectivity for propanol and butanol (e.g., combined selectivity of propanol and butanol of at least 50%), which can be advantageous for the production of propylene and butylene.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

FIG. 1: Schematic of an example of the present invention to produce propylene and butylene.

FIG. 2: is graph depicting CO conversion and product selectivity profile for batch 1 of powdered β-CoMoO₄.

FIG. 3: is graph depicting CO conversion and product selectivity profile for batch 2 of powdered β-CoMoO₄.

FIG. 4: is a graph depicting CO conversion and product selectivity profile for α-CoMoO₄ in powdered form.

FIG. 5: is a graph depicting CO conversion and product selectivity profile for α-CoMoO₄ in pellet form.

FIG. 6: is a graph depicting CO conversion and product selectivity profile for batch 1 of β-CoMoO₄ in pellet form.

FIG. 7: is a graph depicting CO conversion and product selectivity profile for batch 2 of β-CoMoO₄ in pellet form.

FIG. 8: is a graph depicting CO conversion and product selectivity profile for batch 3 of β-CoMoO₄ in pellet form.

### DETAILED DESCRIPTION OF THE INVENTION

A discovery has been made that provides a solution to at least some of the problems associated with the production of propylene and butylene. The solution is premised on using C1 hydrocarbon feedstock to produce these olefins. By way of example, CO and H₂ can be produced from methane. The CO can by hydrogenated with the produced (or supplemental) H₂ using a CO hydrogenation catalyst to produce propanol and butanol with high selectivity. The propanol and butanol can be dehydrated to produce propylene and butylene. It was surprisingly found that a cobalt/molybdenum catalyst having a β-phase crystal structure, such as β-CoMoO₄, exhibits improved syngas conversion and propanol and butanol selectivity compared to a cobalt/molybdenum catalyst having a α-phase crystal structure, such as α-CoMoO₄. Conventional catalyst preparation and processing, specifically, post-calcination grinding or pelletization, can induce a phase change of β-CoMoO₄ to α-CoMoO₄. A method has been discovered for the preparation of a cobalt/molybdenum catalyst that maintains a β-phase crystal structure during work-up and processing.

These and other non-limiting aspects of the present invention are discussed in further detail in the following paragraphs with reference to the figures.

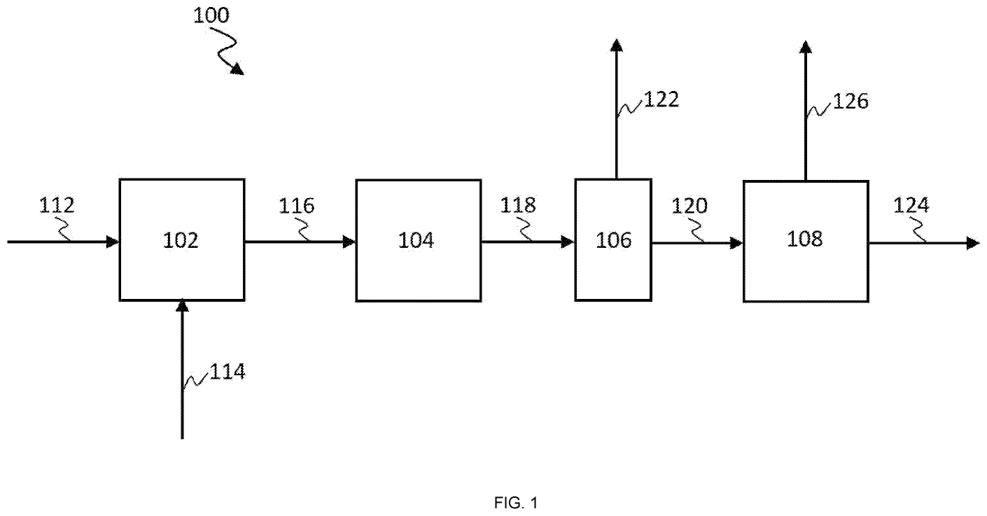

Referring to FIG. 1, one example of a system and process of the present invention for producing propylene and butylene is described. System 100 can include a methane oxidizing unit 102, a CO hydrogenation unit 104, a separation unit 106, and a dehydration unit 108 (e.g., an alcohol dehydration unit).

A first stream 112 containing methane can be fed to the methane oxidizing unit 102. In the methane oxidizing unit 102 the methane can get oxidized by an oxidant to produce syngas (CO and H₂). The oxidant can be steam, O₂, CO₂, or any combination thereof. The oxidant can be fed to the methane oxidizing unit 102 as a separate feed 114 or it can be mixed with the first stream 112 and fed to the methane oxidizing unit 102 as a single feed (not shown). The methane oxidation conditions in the methane oxidizing unit 102 can include: (1) a pressure of 0 bar to 180 bar or at least any one of, equal to any one of, or between any two of 0 bar, 15 bar, 30 bar, 45 bar, 60 bar, 75 bar, 90 bar, 105 bar, 120 bar, 135 bar, 150 bar, 165 bar and 180 bar; (2) a gas hour space velocity (GHSV) of 5000 h⁻¹ to 15000 h⁻¹ or at least any one of, equal to any one of, or between any two of GHSV of 5000 h⁻¹, 6000 h⁻¹, 7000 h⁻¹, 8000 h⁻¹, 9000 h⁻¹, 10000 h⁻¹, 11000 h⁻¹, 12000 h⁻¹, 13000 h⁻¹, 14000 h⁻¹ and 15000 h⁻¹; and/or (3) a temperature of 500° C. to 1600° C. or at least any one of, equal to any one of, or between any two of 500° C., 600° C., 700° C., 800° C., 900° C., 1000° C., 1100° C., 1200° C., 1300° C., 1400° C., 1500° C. and 1600° C. In some aspects, the methane oxidizing unit 102 can contain a methane oxidation catalyst (not shown) and the methane oxidation can be catalyzed by the methane oxidation catalyst. In some aspects, the methane oxidizing unit 102 can be a part of a chemical looping system (not shown), and the methane can be oxidized via chemical looping, wherein the oxidant can be provided to the methane by an oxidized methane oxidation catalyst and/or oxygen transfer agent. The methane oxidation catalyst can contain one or more metals on a support. The one or more metals can be one or more of La, Ni, Ru, Rh, Pd, Ir or Pt, or any alloy, oxide, or combination thereof. The support can be alumina, silica, zirconia, ceria, titania, magnesium oxide, magnesium aluminate, or any combination thereof. In some aspects, the methane oxidation catalyst can contain a promoter. In some aspects the promoter can be an alkali metal, and/or an alkaline earth metal. In some aspects the promoter can be Li, Na, or K, or any alloy, oxide, or combination thereof. Non-limiting examples of methane oxidation catalysts that can be used in the context of the present invention can include LaNiAl₂O₃, LiLaNiAl₂O₃, NaLaNiAl₂O₃, KLaNiAl₂O₃, or a methane oxidation catalyst as described in Khalesi et. al., Ind. Eng. Chem. Res., 2008, 47, 5892-5898.

A second stream 116 containing at least a portion of the CO and H₂ produced from methane oxidation can enter the CO hydrogenation unit 104. The H₂ and CO molar ratio in the second stream can be 0.5:1 to 3:1 or at least any one of, equal to any one of, or between any two of 0.5:1, 0.8:1, 1:1, 1.2:1, 1.5:1, 2:1, 2.5:1, and 3:1. In the CO hydrogenation unit 104 the second stream 116 can be contacted with a CO hydrogenation catalyst (not shown) to hydrogenate the CO with the H₂ and produce propanol, butanol, C2-C7 paraffins, methane and CO₂. The combined selectivity of the propanol and butanol can be 50% to 70% or at least any one of, equal to any one of, or between any two of 50%, 51%, 52%, 53%, 54%, 55%, 56%, 57%, 58%, 59%, 60%, 61%, 62%, 63%, 64%, 65%, 66%, 67%, 68%, 69%, and 70%. In some particular aspects, the selectivity of the propanol can be 12% to 25% or at least any one of, equal to any one of, or between any two of 12%, 13%, 14%, 15%, 16%, 17%, 18%, 19%, 20%, 21%, 22%, 23%, 24%, and 25%. In some particular aspects, the selectivity of the butanol can be 20% to 45% or at least any one of, equal to any one of, or between any two of 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, and 45%. In some particular aspects, the selectivity of the C2 to C7 paraffins can be 30% to 45% or at least any one of, equal to any one of, or between any two of 30%, 31%, 32%, 33%, 34%, 35%, 36%, 37%, 38%, 39%, 40%, 41%, 42%, 43%, 44%, and 45%. In some particular aspects, the selectivity of the CO₂ can be 0% to 5% or at least any one of, equal to any one of, or between any two of 0%, 1%, 2%, 3%, 4%, and 5%. In some particular aspects, the selectivity of the methane can be 3% to 10% or at least any one of, equal to any one of, or between any two of 3%, 4%, 5%, 6%, 7%, 8%, 9%, and 10%. In some aspects, the CO conversion can be 20% to 40% or at least any one of, equal to any one of, or between any two of 20%, 21%, 22%, 23%, 24%, 25%, 26%, 27%, 28%, 29%, 30%, 31%, 32%, 33%, 34%, 35%, 36%, 37%, 38%, 39% and 40%. The CO hydrogenation conditions can include a pressure 25 bar to 90 bar or at least any one of, equal to any one of, or between any two of 25 bar, 35 bar, 45 bar, 55 bar, 65 bar, 75 bar, 85 bar, and 90 bar, GHSV 1000 h⁻¹ to 3000 h⁻¹ or at least any one of, equal to any one of, or between any two of 1000 h⁻¹, 1100 h⁻¹, 1200 h⁻¹, 1300 h⁻¹, 1400 h⁻¹, 1500 h⁻¹, 1600 h⁻¹, 1700 h⁻¹, 1800 h⁻¹, 1900 h⁻¹, 2000 h⁻¹, 2100 h⁻¹, 2200 h⁻¹, 2300 h⁻¹, 2400 h⁻¹, 2500 h⁻¹, 2600 h⁻¹, 2700 h⁻¹, 2800 h⁻¹, 2900 h⁻¹, and 3000 h⁻¹, and/or a temperature 150° C. to 450° C. or at least any one of, equal to any one of, or between any two of 150° C., 200° C., 250° C., 300° C., 350° C., 400° C., and 450° C. In some aspects, the CO hydrogenation catalyst can be activated, prior to contacting the catalyst with the second stream 116. In some aspects, the CO hydrogenation catalyst can be contacted with a stream containing H₂ at a temperature 200° C. to 500° C. or at least any one of, equal to any one of, or between any two of 200° C., 250° C., 300° C., 350° C., 400° C., 450° C. and 500° C., at a GHSV 1000 h⁻¹ to 3000 h⁻¹ or at least any one of, equal to any one of, or between any two of 1000 h⁻¹, 1100 h⁻¹, 1200 h⁻¹, 1300 h⁻¹, 1400 h⁻¹, 1500 h⁻¹, 1600 h⁻¹, 1700 h⁻¹, 1800 h⁻¹, 1900 h⁻¹, 2000 h⁻¹, 2100 h⁻¹, 2200 h⁻¹, 2300 h⁻¹, 2400 h⁻¹, 2500 h⁻¹, 2600 h⁻¹, 2700 h⁻¹, 2800 h⁻¹, 2900 h⁻¹, and 3000 h⁻¹, and/or at a pressure 25 bar to 90 bar or at least any one of, equal to any one of, or between any two of 25 bar, 35 bar, 45 bar, 55 bar, 65 bar, 75 bar, 85 bar, and 90 bar for 8 h to 20 h at least any one of, equal to any one of, or between any two of 8 h, 10 h 12 h, 14 h, 16 h, 18 h and 20 h to reduce and activate the catalyst. In some aspects, the system 100 can include an off-line secondary CO hydrogenation reactor (not shown) in addition to the on-line primary CO hydrogenation reactor 104. The CO hydrogenation catalyst can be activated and/or regenerated in the secondary CO hydrogenation reactor. Activation and/or regeneration of the CO hydrogenation catalyst in the secondary CO hydrogenation reactor can be performed in parallel to the CO hydrogenation in the primary CO hydrogenation reactor 104. Once regeneration/activation of the CO hydrogenation catalyst in the primary CO hydrogenation reactor becomes necessary, the primary CO hydrogenation reactor can be taken off line and the secondary CO hydrogenation reactor with the activated catalyst can be brought on-line and thereby primary become secondary and the secondary becomes primary CO hydrogenation reactor. The parallel activation process can be repeated to ensure continuous operation of the ethylene production process.

The CO hydrogenation catalyst can include a cobalt molybdenum catalyst having a β-phase crystal structure. In some aspects, the CO hydrogenation catalyst can comprise a cobalt molybdenum oxide having a β-phase crystal structure. In some aspects, the CO hydrogenation catalyst can comprise a calcined composition comprising: β-CoₓMo_{y}O_{z}, where x can be 0.5 to 1.5 or at least any one of, equal to any one of, or between any two of 0.5, 0.6, 0.7, 0.8, 0.9, 1, 1.1, 1.2, 1.3, 1.4 and 1.5, y can be 0.5 to 1.5 or at least any one of, equal to any one of, or between any two of 0.5, 0.6, 0.7, 0.8, 0.9, 1, 1.1, 1.2, 1.3, 1.4 and 1.5, and z can balance the valencies of Co and Mo. In certain aspects, z can be 3.5 to 4.5 or at least any one of, equal to any one of, or between any two of 3.5, 3.6, 3.7, 3.8, 3.9, 4, 4.1, 4.2, 4.3, 4.4 and 4.5. In some aspects, the calcined composition can be essentially free of beta-molybdenum carbide (β-Mo₂C), an alkaline metal promoter, and an alkaline earth metal promoter. In some particular aspects, the calcined composition can comprise β-CoMoO₄. In some aspects, the cobalt molybdenum catalyst having a β-phase crystal structure can be prepared using a method including the steps of preparing a solution comprising a cobalt salt and a molybdenum salt and collecting a precipitate from the solution; drying the precipitate to give a dried precipitate comprising one or more hydrates of cobalt molybdenum oxide; optionally pelleting the dried precipitate to produce pellets; and calcining the dried precipitate or optionally the pellets to generate the β-phase catalyst. In some aspects, the pellets are not subjected to mechanical deformation such as grinding subsequent to calcination. The cobalt salt can be cobalt acetate and the molybdenum salt can be ammonium heptamolybdate. In some aspects, the solution contains a binary solvent, preferably ethanol and water, more preferably from 10 to 30% ethanol and from 70 to 90% water, even more preferably 20% ethanol and 80% water, vol:vol. In some aspects, precipitate is dried at a temperature ranging from 70 to 150° C., preferably from 90 to 130° C., more preferably from 100 to 120° C. In some aspects, the precipitate is dried for a period of time ranging from 4 to 8 hours, preferably from 5 to 7 hours. In some embodiments, the pellets are calcined at a temperature ranging from 300 to 700° C., preferably from 400 to 600° C., more preferably from 450 to 550° C. In some aspects, the pellets are calcined for a period of time ranging from 2 to 6 hours, preferably from 3 to 5 hours, more preferably from 2.5 to 3.5 hours. In some aspects, the pellets are calcined under an ambient air environment. Ambient air is defined as atmospheric air present at the calcination unit. In further embodiments, the pellets are calcined under oxygen, nitrogen, helium, or a combination thereof. In other aspects of the invention, however, other CO hydrogenation catalysts can be used.

A third stream before separation 118 containing at least a portion of the propanol, butanol, C2-C7 paraffins, methane, and CO₂ obtained from CO hydrogenation unit 104 can enter the separation unit 106. In some aspects, the stream 118 can contain at least any one of, equal to any one of, or between any two of 12 mol. %, 13 mol. %, 14 mol. %, 15 mol. %, 16 mol. %, 17 mol. %, 18 mol. %, 19 mol. %, 20 mol. %, 21 mol. %, 22 mol. %, 23 mol. %, 24 mol. % and 25 mol. %, propanol; at least any one of, equal to any one of, or between any two of 20 mol. %, 21 mol. %, 22 mol. %, 23 mol. %, 24 mol. %, 25 mol. %, 26 mol. %, 27 mol. %, 28 mol. %, 29 mol. %, 30 mol. %, 31 mol. %, 32 mol. %, 33 mol. %, 34 mol. %, 35 mol. %, 36 mol. %, 37 mol. %, 38 mol. %, 39 mol. %, 40 mol. %, 41 mol. %, 42 mol. %, 43 mol. %, 44 mol. %, and 45 mol. %, butanol; at least any one of, equal to any one of, or between any two of 30 mol. %, 31 mol. %, 32 mol. %, 33 mol. %, 34 mol. %, 35 mol. %, 36 mol. %, 37 mol. %, 38 mol. %, 39 mol. %, 40 mol. %, 41 mol. %, 42 mol. %, 43 mol. %, 44 mol. %, and 45 mol. % C2-C7 paraffins, at least any one of, equal to any one of, or between any two of 3 mol. %, 4 mol. %, 5 mol. %, 6 mol. %, 7 mol. %, 8 mol. %, 9 mol. %, and 10 mol. % methane, and at least any one of, equal to any one of, or between any two of 0 mol. %, 1 mol. %, 2 mol. %, 3 mol. %, 4 mol. %, and 5 mol. % CO₂. In the separation unit 106, the C2-C7 paraffins, methane, and CO₂ can be separated from propanol and butanol. The third stream after separation 120 containing the propanol and butanol can enter the dehydration unit 108 from the separation unit 106 and a stream 122 containing the C2-C7 paraffins, methane, and CO₂ can exit the separation unit 106. The separation of the C2-C7 paraffins, methane, and CO₂ from the third stream in the separation unit 106 can be obtained by any suitable methods known in the art e.g., distillation, fractionation, pressure swing adsorption, and the like. In some aspects, the separation unit 106 can contain a distillation column and the stream 120 can be obtained as a bottom distillate product and the stream 122 can be obtained as a top distillate product. In some aspects, column operating conditions can include a pressure 0 bar to 5 bar or at least any one of, equal to any one of, or between any two of 0 bar, 1 bar, 2 bar, 3 bar, 4 bar and 5 bar and/or a temperature 25° C. to 35° C. or at least any one of, equal to any one of, or between any two of 25° C., 26° C., 27° C., 28° C., 29° C., 30° C., 31° C., 32° C., 33° C., 34° C. and 35° C. In some aspects, at least a portion of methane from stream 122 can be recycled to the methane oxidizing unit 102 (not shown).

The stream 120 can enter the dehydration unit 108. In the dehydration unit 108 the stream 120 can be contacted with an alcohol dehydration catalyst (not shown) under conditions suitable to dehydrate at least a portion of the propanol and butanol and produce a products stream 124 containing propylene and butylene. The dehydration conditions can include: (1) a pressure 0 bar to 90 bar or at least any one of, equal to any one of, or between any two of 0 bar, 10 bar, 20 bar, 30 bar, 40 bar, 50 bar, 60 bar, 70 bar, 80 bar, and 90 bar; (2) a GHSV 1000 h⁻¹ to 3000 or at least any one of, equal to any one of, or between any two of 1000 h⁻¹, 1500 h⁻¹, 2000 h⁻¹, 2500 h⁻¹ and 3000 h⁻¹, and/or (3) a temperature 105° C. to 450° C. or at least any one of, equal to any one of, or between any two of 105° C., 150° C., 200° C., 250° C., 300° C., 350° C., 400° C., and 450° C. The dehydration catalysts can be an acid type catalyst. In some aspects, the acid type catalyst can be cesium doped silicotungstic acid supported on alumina. Non-limiting examples of dehydration catalysts that can be used in the context of the present invention include one or more of CeSiW₁₂O₄₀, RbSiW₁₂O₄₀, CePMo₁₂O₄₀, RbH₃PMo₁₂O₄₀, or a dehydration catalyst as described in Haider et al., Journal of Catalysis 286 (2012) 206-213.

In FIG. 1, the reactors, units and/or zones can include one or more heating and/or cooling devices (e.g., insulation, electrical heaters, jacketed heat exchangers in the wall) and/or controllers (e.g., computers, flow valves, automated values, inlets, outlets, etc.) that can be used to control the reaction temperature and pressure of the reaction mixture. While only one unit or zone is shown, it should be understood that multiple reactors or zones can be housed in one unit or a plurality of reactors housed in one heat transfer unit. In some aspects, the reactors can be a fixed bed reactor, moving bed reactors, trickle-bed reactor, rotating bed reactor, slurry reactors or fluidized bed reactor.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

### EXAMPLES

As part of the disclosure of the present invention, specific examples are included below. The examples are for illustrative purposes only and are not intended to limit the invention. Those of ordinary skill in the art will readily recognize parameters that can be changed or modified to yield essentially the same results.

### Example 1

### Catalyst Preparation

### Example 1A: β-CoMo Powder Preparation

Separate solutions (each in 100 ml of a binary solvent; 80% H₂O, 20% EtOH) of cobalt acetate (12.45 g) and ammonium heptamolybdate (8.45 g) were heated to 65° C. to dissolve the salts. The molybdenum solution was heated at 65° C. while stirring, and the cobalt solution was added dropwise using a separatory funnel. The combined solution was aged for 2 h. The solution was then filtered without washing and the dark purple precipitate was dried in an oven (110° C.) for 6 h. The dried catalyst precursor was ground to a powder then calcined (500° C., static air, 10° C./min heating rate, 4 h). The powder was sieved through 180 micron sieve before testing. The purple color was maintained after calcination. The calcined catalyst (6 ml volume comprising 3 ml catalyst and 3 ml SiC) was then reduced in situ (16 h, H₂, 50 ml/min, 350° C., 1° C. min⁻¹). Two batches, batch 1 (B1) and batch 2 (B2) were than tested to asses reproducibility.

### Example 1B: α-CoMoO₄ Powder and Pellet Preparation

Separate solutions (each in 100 ml of a binary solvent; 80% H₂O, 20% EtOH) of cobalt acetate (12.45 g) and ammonium heptamolybdate (8.45 g) were heated to 65° C. to dissolve the salts. The molybdenum solution was heated at 65° C. while stirring, and the cobalt solution was added dropwise using a separatory funnel. The combined solution was aged for 2 h. The solution was then filtered without washing and the dark purple precipitate was dried in an oven (110° C.) for 6 h. The dried catalyst precursor was ground to a powder then calcined (500° C., static air, 10° C./min, 4 h). The calcined powder was then grinded. Post-calcination grinding induced a phase change from β-CoMoO₄ (purple) to α-CoMoO₄ (green). The color and phase change were observed before loading the green α-CoMoO₄ into the reactor. An in situ pre-reduction H₂ step was performed before syngas testing. The power obtained with post-calcination grinding was then pelleted (10 ton pressure). The pellets were sieved through 200-425 micron sieve before testing.

### Example 1C: β-CoMo Pellet Preparation

In order to confirm that the catalyst prepared in Example 1 is stable in pelleted form and does not change phase upon pelleting, a pelleted version of the Example 1 catalyst (Example 3) was prepared. After preparing the Example 1 catalyst powder described above, the powder was then pelleted (10 ton pressure) then calcined (500° C., static air, 10° C./min, 4 h) to give the final stable pelleted β-CoMoO₄ catalyst. Preparing the catalyst pellets before calcination (when catalyst exists as hydrated form of the β-CoMoO₄) ensured that the catalyst remained in the β-form. The pellets were sieved through 200-425 micron sieve before testing.

### Example 2

### Catalyst Activity/Selectivity Evaluation

The catalysts produced in Examples 1A-C were evaluated for the activity and selectivity, as well as short- and long-term stabilities. Prior to activity measurement, all of the catalysts were subjected to a reductive activation procedure (H₂, 100 ml/min, 350° C., 1° C./min, 16 h). Catalyst evaluation was carried out in a high-throughput, fixed-bed flow reactor setup housed in temperature-controlled system fitted with regulators to maintain pressure during reactions. The products of the reactions were analyzed through online GC analysis. The evaluation was carried out under the following conditions unless otherwise indicated: 75 bar, 300° C., 1° C./min, 48 h stabilization, 100 ml/min, 50% SiC mix. The mass balances of the reactions were calculated to be 95±5%.

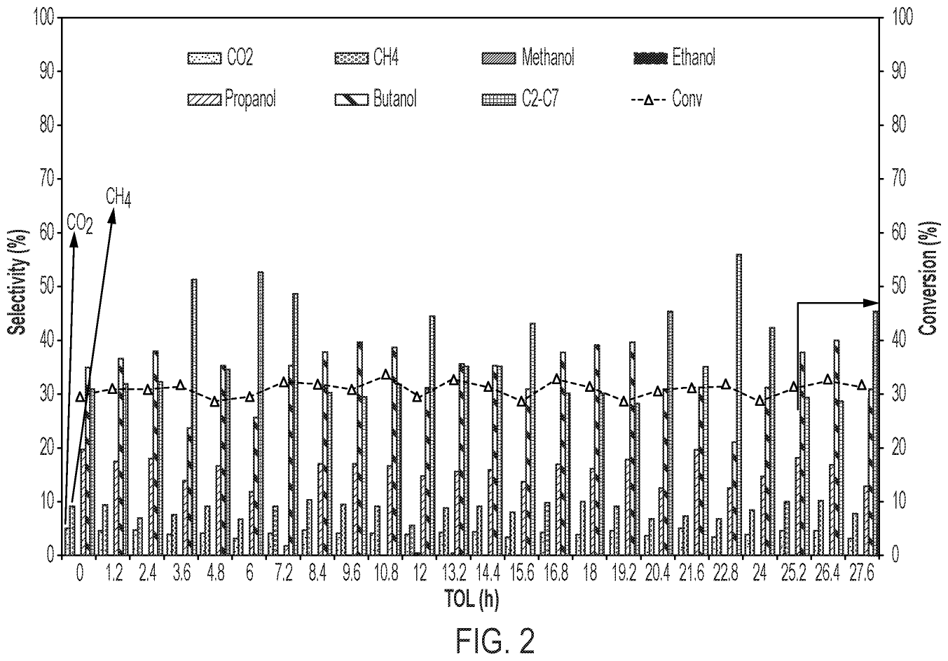

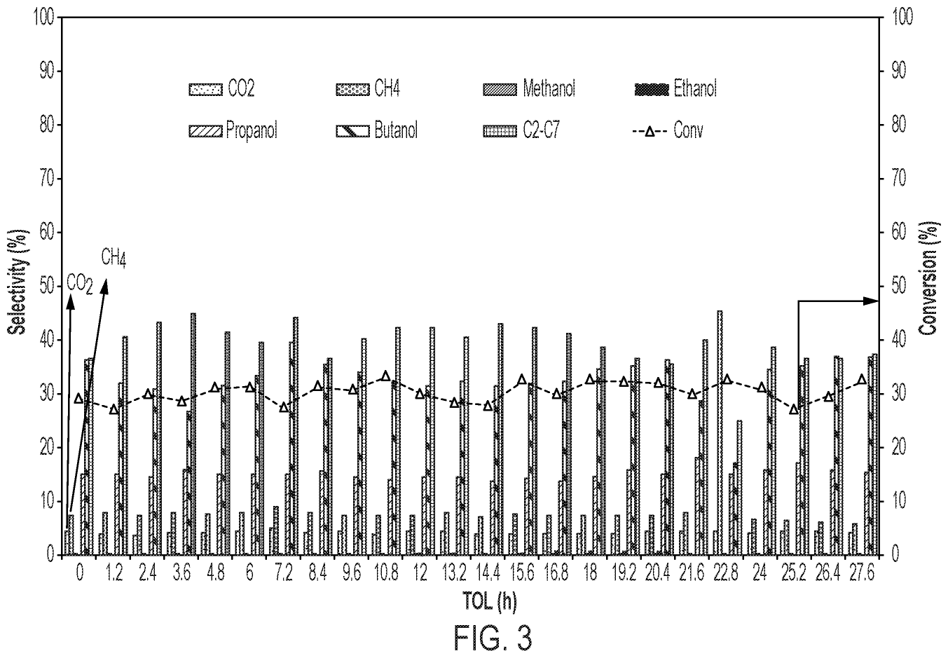

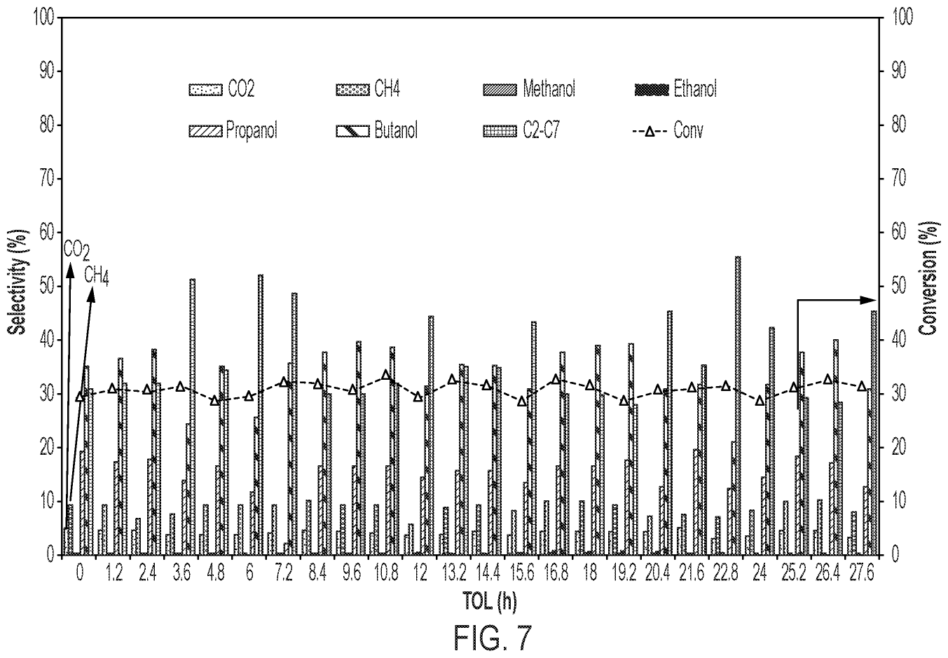

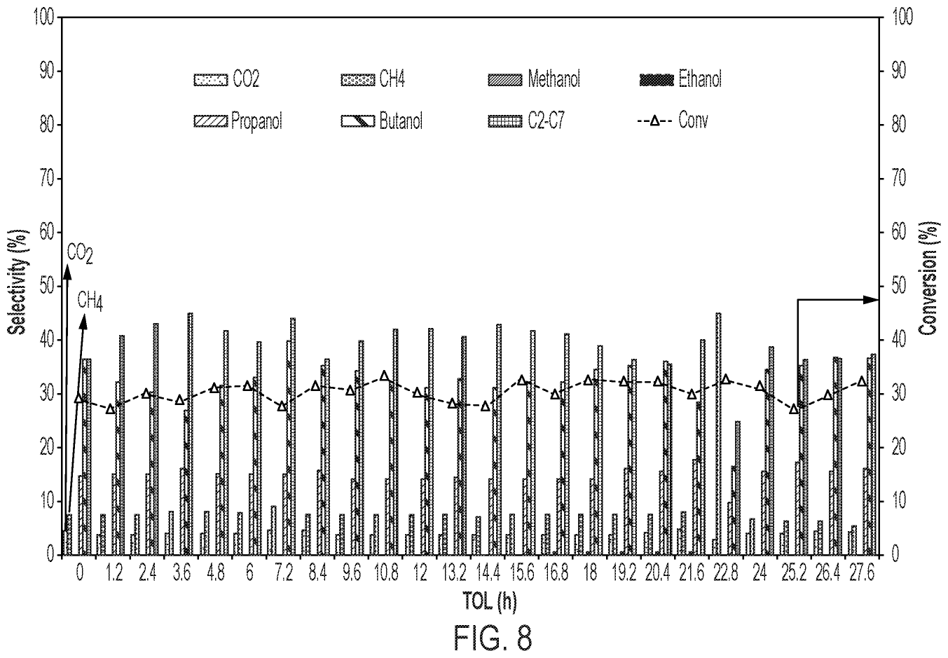

Catalyst testing results are depicted in FIGS. 2-8. FIGS. 2-3 provide results for two catalyst batches prepared in powder form without pelleting, i.e., the β-phase. Cumulative selectivity towards C₃-C₄ alcohols was in the range of 50-60%, with approximately 30% conversion.

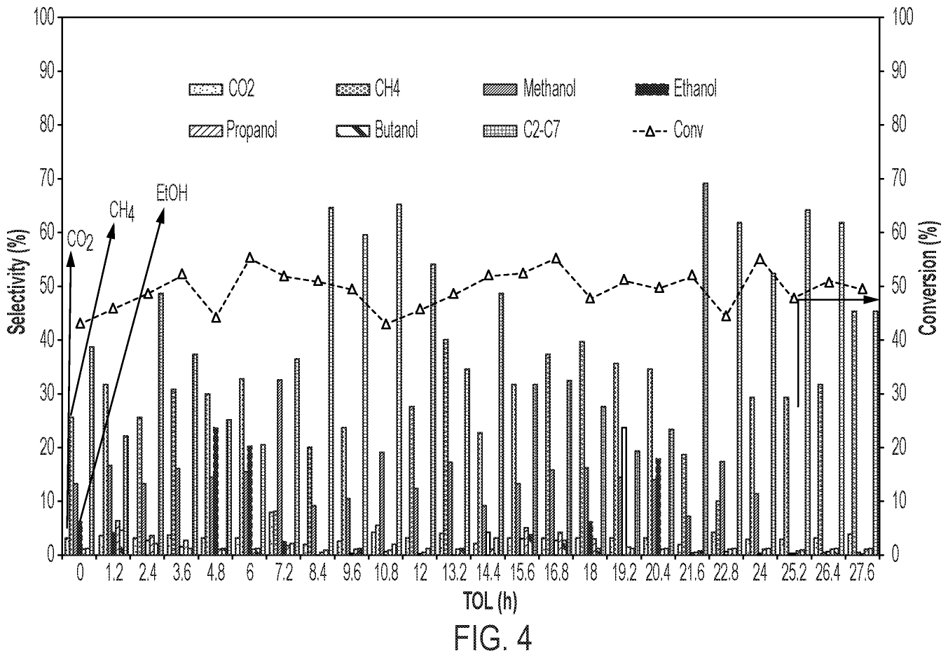

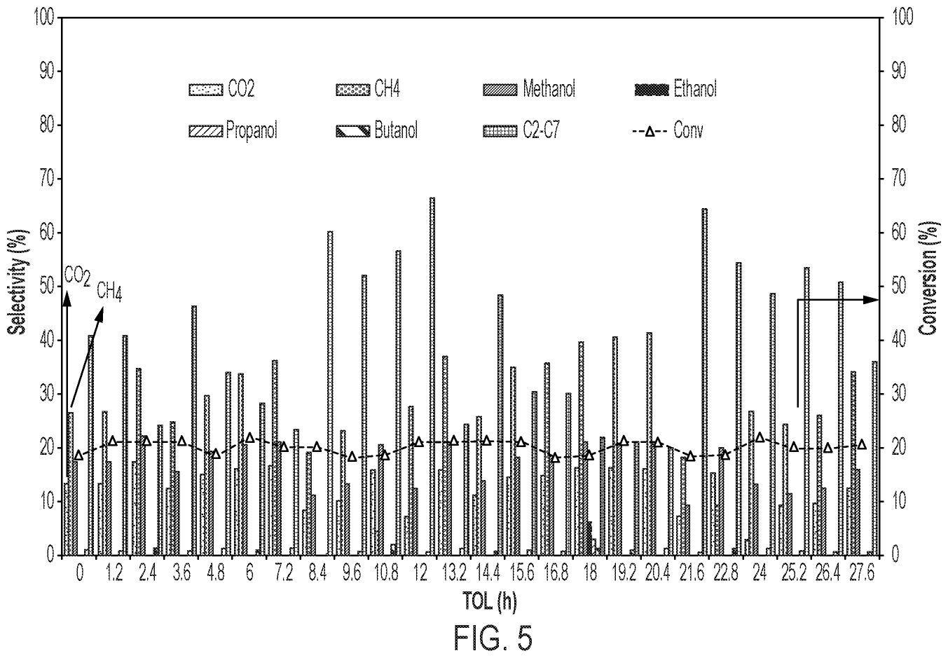

When the catalyst is pelleted/ground post-calcination, the product distribution changes, with methane, methanol, and other hydrocarbons observed as major products (FIGS. 4-5). The distinct product distribution was attributed to the α-CoMoO₄ phase, which was green in color. The results demonstrate that the β-phase catalyst is vastly superior for the production of C₃-C₄ alcohols.

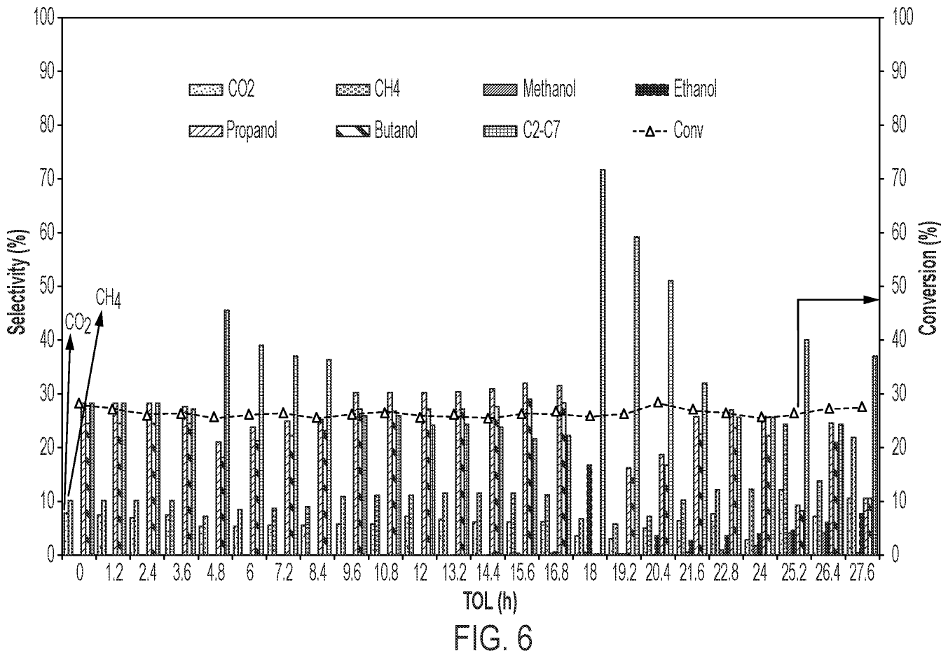

In order to make the catalyst industrially applicable, robust catalytic material must be produced that will endure the harsh conditions provided by fixed bed reactor setups. This goal was achieved by pelleting the catalyst before calcination (in hydrated form). The catalyst (Example 1C) was purple in color and successfully retained the β-CoMoO₄ phase. The results were examined for three batches (FIGS. 6-8). When β-CoMoO₄ was pelleted before calcination, it retained high selectivity for C₃-C₄ alcohols. Cumulative selectivity towards C₃-C₄ alcohols was in the range of 50-60%, however, butanol selectivity was higher for β-pellets (Example 1C, FIGS. 6-8) than for β-powders (Example 1A, FIGS. 2-3). Syngas conversion for β-pellets and β-powders was similar, with conversion amounts at approximately 30%.

It is evident from the data provided herein that the β-CoMoO₄ provides higher selectivity towards propanol and butanol, whereas α-CoMoO₄ catalyst produces more methanol and CO₂. Upon further extending the process to dehydration, metal doped heteropoly acids like silicotungstic acid doped with cesium supported on alumina or silica may be used to produce propylene and butylene in high yields.

In the context of the present invention at least the following 20 embodiments are described. Embodiment 1 is a process for producing propylene and butylene. The process includes: (a) contacting a first stream containing methane with an oxidant and oxidizing at least a portion of the methane under conditions suitable to produce a second stream containing carbon monoxide (CO) and hydrogen (H₂); (b) contacting the second stream with a CO hydrogenation catalyst under conditions suitable to produce a third stream containing propanol and butanol; and (c) contacting the third stream with a dehydration catalyst under conditions suitable to dehydrate at least a portion of the propanol and butanol and produce a products stream containing propylene and butylene. Embodiment 2 is the process of embodiment 1, wherein the third stream further contains C2-C7 paraffins, methane, and carbon dioxide (CO₂) and at least a portion of the C2-C7 paraffins, methane, and carbon dioxide (CO₂) is separated from the third stream before contacting the third stream with the dehydration catalyst. Embodiment 3 is the process of either of embodiments 1 or 2, wherein the CO hydrogenation catalyst includes a cobalt molybdenum containing catalyst having a β-phase crystal structure. Embodiment 4 is the process of embodiment 3, wherein the cobalt molybdenum containing catalyst includes a cobalt molybdenum oxide having a β-phase crystal structure. Embodiment 5 is the process of embodiment 4, wherein the CO hydrogenation catalyst includes a calcined composition containing: β-CoₓMo_{y}O_{z}, wherein x ranges from 0.5 to 2.0, y ranges from 0.5 to 2.0, and z ranges from 3.5 to 4.5. Embodiment 6 is the process of embodiment 5, wherein the calcined composition is essentially free of beta-molybdenum carbide (β-Mo₂C), an alkaline metal promoter, and an alkaline earth metal promoter. Embodiment 7 is the process of either of embodiments 5 or 6, wherein the calcined composition contains β-CoMoO₄. Embodiment 8 is the process of any one of embodiments 1 to 7, wherein the CO hydrogenation catalyst is prepared using a method including preparing a solution containing a cobalt salt and a molybdenum salt and collecting a precipitate from the solution and drying the precipitate to give a dried precipitate containing one or more hydrates of cobalt molybdenum oxide. The method further includes optionally pelleting the dried precipitate to produce pellets, and calcining the dried precipitate or optionally the pellets to generate the CO hydrogenation catalyst, wherein the pellets are optionally not subjected to mechanical deformation subsequent to calcination. Embodiment 9 is the process of any one of embodiments 1 to 8, wherein the CO hydrogenation catalyst is reduced and activated prior to contacting with the second stream. Embodiment 10 is the process of any one of embodiments 1 to 9, wherein the oxidant is steam, oxygen (O₂), CO₂, or a combination thereof. Embodiment 11 is the process of any one of embodiments 1 to 10, wherein the oxidation of the at least a portion of the methane is catalyzed using a methane oxidation catalyst, wherein the methane oxidation catalyst contains one or more metals of La, Ni, Ru, Rh, Pd, Ir, or Pt, on a support containing alumina, silica, zirconia, ceria, titania, magnesium oxide, or magnesium aluminate, or any combination thereof. Embodiment 12 is the process of any one of embodiments 1 to 11, wherein in step (a) the methane oxidation conditions include a pressure of 0 to 180 bar, GHSV of 5000 to 15000 and a temperature of 500 to 1600° C. Embodiment 13 is the process of any one of embodiments 1 to 12, wherein the molar ratio of the H₂ and CO in the second stream is 0.5:1 to 3:1. Embodiment 14 is the process of any one of embodiments 1 to 13, wherein the step (b) contacting conditions include a pressure of 50 to 100 bar, GHSV of 1000 to 3000 and a temperature of 150 to 450° C. Embodiment 15 is the process of any one of embodiments 1 to 14, wherein in step (b) the CO conversion is 25% to 35%, propanol selectivity is 12% to 25%, and butanol selectivity is 20% to 45%. Embodiment 16 is the process of any one of embodiments 2 to 15, wherein the at least a portion of the C2-C7 paraffins, methane and carbon dioxide (CO₂) is separated from the third stream by distillation. Embodiment 17 is the process of any one of embodiments 1 to 16, wherein the step (c) contacting conditions includes a pressure of 0 to 90 bar, GHSV of 1000 to 3000 and a temperature of 105 to 450° C. Embodiment 18 is the process of any one of embodiments 1 to 17, wherein the dehydration catalyst is an acid type catalyst. Embodiment 19 is the process of embodiment 18, wherein the acid type catalyst is cesium doped silicotungstic acid supported on alumina. Embodiment 20 is the process of any one of embodiments 1 to 19, wherein the methane in the first stream is obtained from a refinery, petroleum by product, renewable feedstock, or a combination thereof

1. A process for producing propylene and butylene, the process comprising:
(a) contacting a first stream comprising methane with an oxidant and oxidizing at least a portion of the methane under conditions suitable to produce a second stream comprising carbon monoxide (CO) and hydrogen (H₂);
(b) contacting the second stream with a CO hydrogenation catalyst under conditions suitable to produce a third stream comprising propanol and butanol;
(c) contacting the third stream with a dehydration catalyst under conditions suitable to dehydrate at least a portion of the propanol and butanol and produce a products stream comprising propylene and butylene.

2. The process of claim 1, wherein the third stream further comprises C2-C7 paraffins, methane, and carbon dioxide (CO₂) and at least a portion of the C2-C7 paraffins, methane, and carbon dioxide (CO₂) is separated from the third stream before contacting the third stream with the dehydration catalyst.

3. The process of claim 1, wherein the CO hydrogenation catalyst comprises a cobalt molybdenum containing catalyst having a β-phase crystal structure.

4. The process of claim 3, wherein the cobalt molybdenum containing catalyst includes a cobalt molybdenum oxide having a β-phase crystal structure.

5. The process of claim 4, wherein the CO hydrogenation catalyst comprises a calcined composition comprising: β-CoₓMo_{y}O_{z}, wherein x ranges from 0.5 to 2.0, y ranges from 0.5 to 2.0, and z ranges from 3.5 to 4.5.

6. The process of claim 5, wherein the calcined composition is essentially free of beta-molybdenum carbide (β-Mo₂C), an alkaline metal promoter, and an alkaline earth metal promoter.

7. The process of claim 5, wherein the calcined composition comprises β-CoMoO₄.

8. The process of claim 1, wherein the CO hydrogenation catalyst is prepared using a method comprising:
(i) preparing a solution comprising a cobalt salt and a molybdenum salt and collecting a precipitate from the solution;
(ii) drying the precipitate to give a dried precipitate comprising one or more hydrates of cobalt molybdenum oxide;
(iii) optionally pelleting the dried precipitate to produce pellets; and
(iv) calcining the dried precipitate or optionally the pellets to generate the CO hydrogenation catalyst,
wherein the pellets are optionally not subjected to mechanical deformation subsequent to calcination.

9. The process of claim 1, wherein the CO hydrogenation catalyst is reduced and activated prior to contacting with the second stream.

10. The process of claim 1, wherein the oxidant is steam, oxygen (O₂), CO₂, or a combination thereof.

11. The process of claim 1, wherein the oxidation of the at least a portion of the methane is catalyzed using a methane oxidation catalyst, wherein the methane oxidation catalyst comprises one or more metals of La, Ni, Ru, Rh, Pd, Ir, or Pt, on a support comprising alumina, silica, zirconia, ceria, titania, magnesium oxide, or magnesium aluminate, or any combination thereof.

12. The process of claim 1, wherein in step (a) the methane oxidation conditions comprise a pressure of 0 to 180 bar, GHSV of 5000 to 15000 h⁻¹ and a temperature of 500 to 1600° C.

13. The process of claim 1, wherein the molar ratio of the H₂ and CO in the second stream is 0.5:1 to 3:1.

14. The process of claim 1, wherein the step (b) contacting conditions comprise a pressure of 50 to 100 bar, GHSV of 1000 to 3000 h⁻¹, and a temperature of 150 to 450° C.

15. The process of claim 1, wherein in step (b) the CO conversion is 25% to 35%, propanol selectivity is 12% to 25%, and butanol selectivity is 20% to 45%.

16. The process of claim 2, wherein the at least a portion of the C2-C7 paraffins, methane and carbon dioxide (CO₂) is separated from the third stream by distillation.

17. The process of claim 1, wherein the step (c) contacting conditions comprises a pressure of 0 to 90 bar, GHSV of 1000 to 3000 h⁻¹ and a temperature of 105 to 450° C.

18. The process of claim 1, wherein the dehydration catalyst is an acid type catalyst.

19. The process of claim 18, wherein the acid type catalyst is cesium doped silicotungstic acid supported on alumina.

20. The process of claim 1, wherein the methane in the first stream is obtained from a refinery, petroleum by product, renewable feedstock, or a combination thereof.
